(19) United States
(12) Patent Application Publication
(10) Pub. No.: US 2007/0254960 A1
(43) Pub. Date: Nov. 1, 2007

(54) PHARMACEUTICAL COMBINATION

(75) Inventors: Petra Bloms-Funke, Wuerselen (DE); Klaus Schiene, Duesseldorf (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/741,420

(22) Filed: Apr. 27, 2007

Related U.S. Application Data
(60) Provisional application No. 60/795,579, filed on Apr. 28, 2006.

(30) Foreign Application Priority Data
Apr. 28, 2006 (EP) 06008851.5

(51) Int. Cl. A61K 31/137

(52) U.S. Cl. 514/649

(57) ABSTRACT

A combination comprising as components (a) the compound 3-(3-Dimethylamino-1-ethyl-2-methyl-propyl)-phenol, and (b) Paracetamol or a derivative thereof, a pharmaceutical formulation and a dosage form comprising said combination as well as a method of treating pain, e.g. chronic or acute pain, characterized in that components (a) and (b) are administered simultaneously or sequentially to a mammal, wherein component (a) may be administered before or after component (b) and wherein components (a) or (b) are administered to the mammal either via the same or a different pathway of administration.

### CROSS-REFERENCE TO RELATED APPLICATIONS

This application is claims benefit to U.S. provisional patent application Ser. No. 60/795,579 filed Apr. 28, 2006 and European patent application Serial No. EP06008851.5 filed Apr. 28, 2006, the entire disclosures of which are hereby incorporated in their entirety.

### FIELD OF THE INVENTION

The present invention relates to a combination comprising as components (a) the compound 3-(3-Dimethylamino-1-ethyl-2-methyl-propyl)-phenol, and (b) Paracetamol or a derivative thereof, a pharmaceutical formulation and a dosage form comprising said combination as well as a method of treating pain, e.g. chronic or acute pain, characterized in that components (a) and (b) are administered simultaneously or sequentially to a mammal, wherein component (a) may be administered before or after component (b) and wherein components (a) or (b) are administered to the mammal either via the same or a different pathway of administration.

### BACKGROUND OF THE INVENTION

The treatment of chronic and acute pain conditions is extremely important in medicine. There is currently a worldwide demand for additional, not exclusively opioid-based, but highly effective pain treatment. The urgent need for action for patient-oriented and purposeful treatment of pain conditions, this being taken to mean the successful and satisfactory treatment of pain for the patient, is documented in the large number of scientific papers which have recently appeared in the field of applied analgesics and fundamental research work on nociception.

Even if the analgesics that are currently used for treating pain, for example opioids, NA- and 5HT-reuptake inhibitors, NSAIDS and COX inhibitors, are analgesically effective, side effects nevertheless sometimes occur. WO 2004/047823 describes substance combinations comprising certain analgesics including 1-phenyl-3-dimethylamino-propane compounds and COX-II Inhibitors, which show super-additive effects upon administration. Due to the super-additive effect the overall dose and accordingly the risk of undesired side effects can be reduced.

### SUMMARY OF THE INVENTION

Thus, it was an object of the present invention to find further combinations that are suitable for the treatment of pain and which preferably exhibit fewer undesired side effects compared to its individual components, if administered in effective doses.

It has been found that a combination comprising (a) the compound 3-(3-Dimethylamino-1-ethyl-2-methyl-propyl)-phenol, and (b) Paracetamol or a derivative thereof exhibits an analgesic effect. If these components are present in the composition in such a weight ratio that a synergistic effect is observed after administration to the patients, the overall administered dose may be lowered, so that fewer undesired side-effects will occur.

Accordingly, the present invention relates to a pharmaceutical combination comprising as components

(a) 3-(3-Dimethylamino-1-ethyl-2-methyl-propyl)-phenol of formula (I) 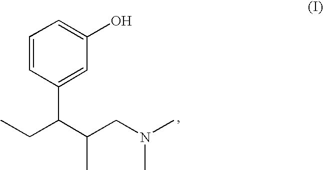
optionally in form of one of its pure stereoisomers, in particular an enantiomer or a diastereomer, a racemate or in form of a mixture of its stereoisomers, in particular enantiomers and/or diastereomers in any mixing ratio, or any corresponding acid addition salt thereof, or any solvate thereof, and

(b) Paracetamol or a derivative thereof.

In an embodiment of the inventive combination component (a) is selected from
(1R,2R)-3-(3-Dimethylamino-1-ethyl-2-methyl-propyl)-phenol,
(1S,2S)-3-(3-Dimethylamino-1-ethyl-2-methyl-propyl)-phenol,
(1R,2S)-3-(3-Dimethylamino-1-ethyl-2-methyl-propyl)-phenol,
(1S,2R)-3-(3-Dimethylamino-1-ethyl-2-methyl-propyl)-phenol, and any mixture thereof.

In another embodiment of the inventive combination component (a) is selected from
(1R,2R)-3-(3-Dimethylamino-1-ethyl-2-methyl-propyl)-phenol, and
(1S,2S)-3-(3-Dimethylamino-1-ethyl-2-methyl-propyl)-phenol, and any mixture thereof.

In yet another embodiment the inventive combination comprises

(a) the compound (1R,2R)-3-(3-Dimethylamino-1-ethyl-2-methyl-propyl)-phenol of formula (I′), 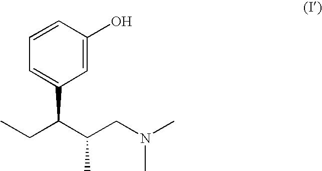
or an acid addition salt thereof, and

(b) Paracetamol or a derivative thereof.

The compound 3-(3-Dimethylamino-1-ethyl-2-methyl-propyl)-phenol of formula (I), its stereoisomers and corresponding salts thereof as well as methods for their preparation are well known, for example, from U.S. Pat. No. 6,248,737 B1. The respective parts of the description are hereby incorporated by reference and form part of the present disclosure.

The definition of component (a) as used herein includes the compound 3-(3-Dimethylamino-1-ethyl-2-methyl-propyl)-phenol and its stereoisomers in any possible form, thereby particularly including solvates, acid addition salts and corresponding solvates and polymorphs thereof.

If component (a) is present as mixture of enantiomers, such a mixture may contain the enantiomers in racemic or non-racemic form. A non-racemic form could, for example, contain the enantiomers in a ratio of 60:40, 70:30, 80:20 or 90:10.

The compound 3-(3-Dimethylamino-1-ethyl-2-methyl-propyl)-phenol and its stereoisomers according to component (a) may be present in the inventive pharmaceutical composition in form of an acid addition salt, whereby any suitable acid capable of forming such an addition salt may be used.

The conversion of the compound 3-(3-Dimethylamino-1-ethyl-2-methyl-propyl)-phenol into a corresponding addition salt via reaction with a suitable acid may be effected in a manner well known to those skilled in the art. Suitable acids include but are not limited to hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid and/or aspartic acid. Salt formation is preferably effected in a solvent, for example diethyl ether, diisopropyl ether, alkyl acetates, acetone and/or 2-butanone. Moreover, trimethylchlorosilane in aqueous solution is also suitable for the preparation of hydrochlorides.

It is known to those skilled in the art that the analgesic action of non-steroidal anti-inflammatory drugs (NSAIDs) is due to the inhibition of the enzymatic production of prostaglandins, wherein Cyclooxygenase (COX) is the key enzyme in the conversion of arachidonic acid derived from lipids of the cell membrane to prostaglandins and other eicosanoids. COX exists in two different isoforms characterized by different expression patterns. COX-I is constitutively expressed in many cells of the body and responsible mainly for the production of eicosanoids serving normal physiological functions. COX-II expression is induced during inflammation and also COX-II is expressed in the central nervous system.

Paracetamol and its derivatives do not show any significant anti-inflammatory activity and are accordingly not considered to be NSAIDs.

The term paracetamol, also known as acetaminophen, and its derivatives as used herein includes these compounds in any possible form, thereby including solvates and polymorphs thereof.

The term derivative as used herein particularly includes prodrugs such as ethers and esters of Paracetamol. Suitable methods for selecting and preparing a pro-drug of a given substance are for example described in “Textbook of Drug Design and Discovery”, 3^{rd} edition, 2002, chapter 14, pages 410-458, Editors: Krogsgaard-Larsen et al., Taylor and Francis. The respective parts of said literature description are incorporated by reference and form part of the present disclosure.

Paracetamol and its derivatives such as Propacetamol and Phenidine as well as processes for their preparation are well known in the art, for example from E. Friderichs et al. “Analgesics and Antipyretics”, Ullmann's Encyclopedia of Industrial Chemistry, Sixth Edition, Wiley-VCH Verlag GmbH, Germany 2000, pages 1-22 and H. Buschmann, T. Christoph, E. Friderichs, C. Maul, B. Sundermann, “Analgesics—From Chemistry and Pharmacology to Clinical Application”, 2002, Part II, Wiley-VCH Verlag, Germany. The respective parts of said literature descriptions are incorporated by reference and form part of the present disclosure.

In one embodiment of the inventive combination the derivative of Paracetamol according to component (b) is selected from the group consisting of Propacetamol and Phenidine.

A specific embodiment of the present invention is a combination comprising
(a) (1R,2R)-3-(3-Dimethylamino-1-ethyl-2-methyl-propyl)-phenol, or the hydrochloride addition salt thereof, and (b) Paracetamol.

Both components (a) and (b) as part of the inventive combination may be administered in their usual daily dosage. The daily dosage of paracetamol should preferably not exceed 4 g for adults. For infants and children the daily dosage should preferably not exceed 90 mg/kg. Preferably the compound (1R,2R)-3-(3-Dimethylamino-1-ethyl-2-methyl-propyl)-phenol may be administered to a patient in a daily dosage of 25 to 1000 mg, particularly preferably in a dosage of 50 to 800 mg, more particularly preferably in a dosage of 100 to 600 mg.

In another embodiment of the present invention the inventive combination may contain components (a) and (b) essentially in an equieffective ratio.

In yet a further embodiment of the inventive combination components (a) and (b) are present in such a weight ratio that the resulting composition will exert a synergistic effect upon administration to a patient. Suitable weight ratios can be determined by methods well known to those skilled in the art, e.g. via the Randall-Selitto test described below.

Both components (a) and (b) may also be present in the inventive combination in ratios deviating from the equieffective ratio. For, example, each of the components could be present in a range from 1/5 of the equieffective amount to 5 times the equieffective amount, preferably 1/4 to 4, more preferably 1/3 to 3, yet more preferably 1/2 to 2 of the equieffective amount.

In another embodiment of the present invention the components (a) and (b) can be administered in a specific dosage regimen to treat pain, for example, chronic pain or acute pain. Components (a) and (b) may be administered simultaneously or sequentially to one another, in each case via the same or different administration pathways. Another aspect of the present invention is therefore a method of treating pain, e.g. chronic or acute pain, characterized in that components (a) and (b) are administered simultaneously or sequentially to a mammal, wherein component (a) may be administered before or after component (b) and wherein components (a) or (b) are administered to the mammal either via the same or a different pathway of administration. Suitable pathways of administrations include but are not limited to oral, intravenous, intraperitoneal, transdermal, intrathekal, intramuscular, intranasal, transmucosal, subcutaneous, or rectal administration.

The inventive combinations are toxicologically safe and are therefore suitable for the treatment of mammals, particularly humans including infants, children and grown-ups.

Thus, in a further aspect the present invention relates to a pharmaceutical composition comprising an inventive combination as described herein and one or more auxiliary agents.

In a further aspect the present invention relates to a pharmaceutical dosage form comprising an inventive combination as described herein and one or more auxiliary agents.

In one embodiment the inventive pharmaceutical dosage form additionally comprises caffeine.

In one embodiment, the inventive pharmaceutical dosage form is suitable for being administered orally, intravenously, intraperitoneally, transdermally, intrathekally, intramuscularly, intranasally, transmucosally, subcutaneously, or rectally.

The inventive formulations and dosage forms may contain auxiliary agents, for example, carriers, fillers, solvents, diluents, colorants and/or binders. The selection of auxiliary agents and of the amounts of the same to be used depends, for example, on how the drug is to be administered, e.g. orally, intravenously, intraperitoneally, intradermally, intramuscularly, intranasally or locally, for example for infections of the skin, of the mucous membranes or of the eye.

Suitable auxiliary agents in the context of this invention are any substances known to a person skilled in the art useful for the preparation of galenical formulations. Examples of suitable auxiliary agents include but are not limited to: water, ethanol, 2-propanol, glycerol, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl cellulose, cellulose acetate, shellac, cetyl alcohol, polyvinyl pyrrolidone, paraffins, waxes, natural and synthetic gums, acacia gum, alginates, dextran, saturated and unsaturated fatty acids, stearic acid, magnesium stearate, zinc stearate, glycerol stearate, sodium lauryl sulphate, edible oils, sesame oil, coconut oil, peanut oil, soybean oil, lecithin, sodium lactate, polyoxyethylene and polypropylene fatty acid ester, sorbitan fatty acid ester, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulphate, zinc sulphate, calcium sulphate, potash, calcium phosphate, dicalcium phosphate, potassium bromide, potassium iodide, talcum, kaolin, pectin, crosspovidone, agar and bentonite.

Pharmaceutical formulations (dosage forms) in the form of tablets, effervescent tablets, chewing tablets, dragees, capsules, drops, juices or syrups are, for example, suitable for oral administration. Oral pharmaceutical formulations may also be in the form of multiparticulates such as granules, pellets, spheres, crystals and the like, optionally compressed into a tablet, filled into a capsule, filled into a sachet or suspended in a suitable liquid medium. Oral pharmaceutical formulations may also be equipped with an enteric coating.

Pharmaceutical formulations that are suitable for parenteral, topical and inhalative administration include but are not limited to solutions, suspensions, easily reconstitutable dry preparations and sprays.

Suppositories are a suitable pharmaceutical formulation for rectal administration. Formulations in a deposit, in dissolved form, for example, in a patch optionally with the addition of agents to promote skin penetration, are examples of suitable formulations for percutaneous administration.

One or both of the components (a) and (b) may be present in the inventive pharmaceutical formulation at least partially in controlled-release form. Moreover, any controlled release/immediate release combination of said components may also be present in the inventive pharmaceutical formulation. For example, one or both of the components may be released from the inventive formulations with a certain delay, e.g. if administered orally, rectally or percutaneously. Such formulations are particularly useful for “once-daily” or “twice-daily” preparations, which only have to be taken once a day, respectively, twice a day. Suitable controlled-release materials are well known to those skilled in the art.

The inventive pharmaceutical formulations may be produced using materials, means, devices and processes that are well known in the prior art of pharmaceutical formulations, as described for example in “Remington's Pharmaceutical Sciences”, A. R. Gennaro (ed.), 17^{th} edition, Mack Publishing Company, Easton, Pa. (1985), in particular in part 8, chapters 76 to 93.

In order to obtain a solid pharmaceutical formulation such as a tablet, for example, the components of the pharmaceutical composition may be granulated with a pharmaceutical carrier, for example conventional tablet ingredients such as corn starch, lactose, saccharose, sorbitol, talcum, magnesium stearate, dicalcium phosphate or pharmaceutically acceptable gums, and pharmaceutical diluents, for example water, in order to form a solid composition that contains the components in homogeneous distribution. The term “homogeneous distribution” is taken to mean that the components are distributed uniformly over the entire composition, so that said composition may easily be divided into equally effective unit dose forms, such as tablets, pills or capsules. The solid composition is then divided into unit dose forms. The tablets or pills of the pharmaceutical composition according to the invention may also be coated or compounded in a different manner, in order to provide a dose form with a controlled release.

If one of the components, e.g. component (b), is to be released prior to the other component, for example at least 30 minutes or 1 hour beforehand, pharmaceutical formulations having a corresponding release profile may be prepared. An example of such a formulation is an osmotically driven release system for achieving a delayed release of component (a) via a coating that itself contains component (b) which is accordingly released earlier. In a release system of this kind, which is particularly suitable for oral administration, at least part, and preferably all, of the surface of the release system, preferably those parts that will come into contact with the release medium, is/are semipermeable, preferably equipped with a semipermeable coating, so the surface(s) is/are permeable to the release medium, but substantially, preferably entirely, impermeable to the active ingredient, component (a), the surface(s) and/or optionally the coating comprising at least one opening for releasing the active ingredient, component (a). Moreover, precisely that/those surface(s) that is/are in contact with the release medium is/are provided with a coating containing and releasing the other component, component (b). This is preferably taken to mean a system in tablet form comprising a release opening, an osmotic pharmaceutical composition core, a semipermeable membrane and a polymer portion that exerts pressure upon swelling. A suitable example of this kind of system is the system distributed by ALZA Corporation, USA under the tradenames OROS®, in particular, the OROS® Push-Pull™ System, the OROS® Delayed Push-Pull™ System, the OROS® Multi-Layer Push-Pull™ system, the OROS® Push-Stick System and also, in specific cases, the L-OROS™.

Embodiments and examples of osmotically driven release systems are, for example, disclosed in U.S. Pat. Nos. 4,765,989, 4,783,337 and 4,612,008, all of the respective contents of which are hereby incorporated by reference and form part of the disclosure of the present invention.

A further example of a suitable pharmaceutical formulation is a gel-matrix tablet, such as the products developed by Penwest Pharmaceuticals (for example, under TimeRX). Suitable examples are provided in U.S. Pat. Nos. 5,330,761, 5,399,362, 5,472,711 and 5,455,046, all of the respective contents of which are hereby incorporated by reference and form part of the disclosure of the present invention. Particularly suitable is a retarding matrix formulation, with an inhomogeneous distribution of the pharmaceutically active composition, whereby, for example, the component (b) can be distributed in the outer region (the portion that comes into contact with the release medium most quickly) of the matrix and the other component (a) is distributed inside the matrix. On contact with the release medium, the outer matrix layer initially (and rapidly) swells and firstly releases the Paracetamol component, followed by the significantly (more) retarded release of component (a). Examples of a suitable matrix include matrices with 1 to 80% by weight of one or more hydrophilic or hydrophobic polymers as pharmaceutically acceptable matrix formers. A further example of a suitable matrix may be inferred from U.S. Pat. No. 4,389,393 the respective contents of which hereby being incorporated by reference and forming part of the disclosure of the present invention.

The amount of the inventive pharmaceutically active combination to be administered to the patient may vary depending on different factors well known to those skilled in the art, for example, the weight of the patient, the route of administration, or the severity of the illness.

In a further aspect the present invention relates to the use of an inventive combination as described herein for the preparation of a medicament for the treatment of pain.

In another embodiment the present invention relates to the use of an inventive combination as described herein for the preparation of a medicament for the treatment of pain, wherein the pain is selected from inflammatory pain, neuropathic pain, acute pain, chronic pain, visceral pain, migraine pain and cancer pain.

In yet another aspect the present invention relates to a method of treating pain in a mammal, preferably a human, which comprises administering an effective amount of an inventive combination as described herein to the mammal.

In a further aspect of the present invention it relates to a method of treating pain in a mammal, preferably a human, which comprises administering an effective amount of an inventive combination as described herein to the mammal, wherein the pain is selected from inflammatory pain, neuropathic pain, acute pain, chronic pain, visceral pain, migraine pain and cancer pain.

Pharmacological Methods:

A. Randall-Selitto Test in Rats

The weight ratios of the components (a) and (b) that will lead to a supra-additive effect (synergistic effect) of the inventive pharmaceutical composition may be determined via the test of Randall and Selitto as described in Arch. Int. Pharmacodyn., 1957, 111: 409 to 419, which is a model for inflammatory pain. The respective part of the literature is hereby incorporated by reference and forms part of the present disclosure.

By means of injection of 0.1 ml of Carrageenin-suspension ventrally into a hind paw of a rat an oedema is induced, on which pain is generated 4 hours later by continuously increasing pressure with a stamp (2 mm tip diameter). The antinociceptive and antihyperalgesic activity of the tested substance is determined at different points in time after administration of the substance. The measured value to be determined and at the same time also the end point of the pain test is the pressure at which the vocalisation reaction of the rat occurs. The percentage maximum possible effect (% MPE) is calculated. The maximum pressure of the stamp is 250 g. The group size is n=10.

The analysis of the results with respect to a supra-additive effect of the inventive pharmaceutical composition comprising the components (a) and (b) is carried out via statistical comparison of the theoretical additive ED₅₀-value with the experimentally determined ED₅₀-value of a so-called fixed ratio combination (isobolographic analysis according to Tallarida J T, Porreca F, and Cowan A. Statistical analysis of drug-drug and site-site interactions with isobolograms. Life Sci 1989; 45: 947-961).

The interactions studies presented herein were performed using equieffective doses of the two components, calculated from the ratio of the respective ED₅₀ values of the components if administered alone.

The application route was intravenous (i.v.) for (1R,2R)-3-(3-Dimethylamino-1-ethyl-2-methyl-propyl)-phenol (A) and intraperitoneal (i.p.) for Paracetamol. When A was applied alone, the peak effect was reached 15 min p. appl. (timepoint of first measurement) and an ED₅₀-value of 1.878 (1.694-2.065) mg/kg i.v. was calculated. Paracetamol induced a dose-dependent analgesic effect with an ED₅₀-value of 189.9 (181.3-198.4) mg/kg i. p. respectively, reaching the peak effect 120 min p. appl. According to their respective timepoint of peak effect, (1R,2R)-3-(3-Dimethylamino-1-ethyl-2-methyl-propyl)-phenol was applied 15 min and Paracetamol 120 min before timepoint of measurement of the interaction-experiments (i. e. Paracetamol was applied 105 min before (1R,2R)-3-(3-Dimethylamino-1-ethyl-2-methyl-propyl) -phenol, respectively). Thus, the time point of ED₅₀ calculation of the combination corresponds to the timepoint of the peak effect of the respective compound. The isobolographic analysis revealed that the experimental ED₅₀-values of the combinations were significantly lower than the respective theoretical ED₅₀-values. Thus, the combination studies demonstrate significant synergistic interaction of (1R,2R)-3-(3-Dimethylamino-1-ethyl-2-methyl-propyl)-phenol with Paracetamol.

The results of the isobolographic analysis are summarized in the following table.

Experimental ED₅₀ values of A and Paracetamol and isobolographic analysis of the interaction between A and Paracetamol:

| | | | Theoretical | Experimental | |
|---|---|---|---|---|---|
| | | | ED50 of the | ED50 of the | |
| | | | combination | combination | |
| | | Para- | of A and | of A and | |
| | A | cetamol | Paracetamol | Paracetamol | interaction |
| Sub- | 1.878 | 189.9 | 95.90 (90.75- | 74.88 (66.63- | supra- |
| stance/ | (1.694- | (181.3- | 101.0) | 84.17) | additive |
| ED50 | 2.065)* | 198.4) | | | (p < |
| [mg/kg] | | | | | 0.001) |
| (confi- | | | | | |
| dence | | | | | |
| interval) | | | | | |

p: Level of statistical significance

From table 1 given above, the ratio of A to Paracetamol can be calculated to be 1:101

The following example is provided to illustrate the process according to the invention in greater detail and do not and should not be understood to limit the claims appended hereto. The invention is not limited in its application to the details of any particular formulation shown, since the invention is capable of other embodiments.

### EXAMPLE

Preparation of a Paracetamol combination tablet

Composition

| | | |
|---|---|---|
| Paracetamol | 5000 | g |
| (1R,2R)-3-(3-Dimethylamino-1-ethyl-2-methyl-propyl)-phenol | 500 | g |
| Povidone K25 | 100 | g |
| Microcrystalline Cellulose | 300 | g |
| Powdered Cellulose | 140 | g |
| Stearic Acid | 60 | g |

Povidone is dissolved in 1.5 liter of water. Paracetamol and (1R,2R)-3-(3-Dimethylamino-1-ethyl-2-methyl-propyl)-phenol are blended in a high shear mixer and the compounds are granulated by adding the povidone solution. The wet mass is sized through a 3 mm sieve and dried in an oven at 50° C. The dry mass is sized together with microcrystalline cellulose and powdered cellulose through an 1 mm sieve. The mass is blended together with the stearic acid that has been passed through an 0.315 mm sieve. The final mass is pressed on an Korsch EK0 tablet press into tablets of 13 mm diameter and a weight of 610 mg each.

The foregoing description and example have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A composition comprising:
(a) at least one 3-(3-Dimethylamino-1-ethyl-2-methyl-propyl)-phenol compound corresponding to formula (I), 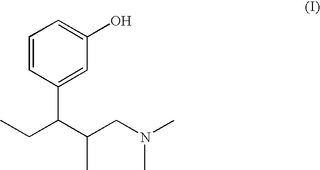
or a solvate or an acid addition salt thereof, and
(b) Paracetamol or a derivative thereof.

2. The composition of claim 1, wherein said compound corresponding to formula (I) is present in the form of a pure enantiomer or pure diastereoisomer.

3. The composition of claim 1, wherein said compound corresponding to formula (I) is present in the form of a mixture of stereoisomers.

4. The composition of claim 1, wherein said compound corresponding to formula (I) is present in the form of a racemic mixture.

5. The composition of claim 1, wherein said compound corresponding to formula (I) is present in the form of a solvate.

6. The composition of claim 1, wherein said compound corresponding to formula (I) is present in the form of an acid addition salt.

7. The composition of claim 1, wherein said compound corresponding to formula (I) is selected from the group consisting of:
(1R,2S)-3-(3-Dimethylamino-1-ethyl-2-methyl-propyl)-phenol,
(1S,2R)-3-(3-Dimethylamino-1-ethyl-2-methyl-propyl)-phenol, and
any mixture of
(1R,2R)-3-(3-Dimethylamino-1-ethyl-2-methyl-propyl)-phenol,
(1S,2S)-3-(3-Dimethylamino-1-ethyl-2-methyl-propyl)-phenol,
(1R,2S)-3-(3-Dimethylamino-1-ethyl-2-methyl-propyl)-phenol, and
(1S,2R)-3-(3-Dimethylamino-1-ethyl-2-methyl-propyl)-phenol.

8. The composition of claim 1, wherein said compound corresponding to formula (I) is selected from the group consisting of:
(1R,2R)-3-(3-Dimethylamino-1-ethyl-2-methyl-propyl)-phenol,
(1S,2S)-3-(3-Dimethylamino-1-ethyl-2-methyl-propyl)-phenol, and any mixture thereof.

9. The composition of claim 1, wherein said compound corresponding to formula (I) is a (1R,2R)-3-(3-Dimethylamino-1-ethyl-2-methyl-propyl)-phenol compound corresponding to formula (I′), 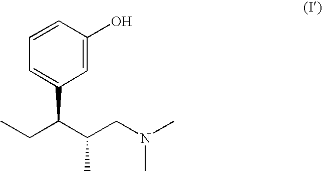
or an acid addition salt thereof.

10. The composition of claim 9, wherein said compound corresponding to formula (I) is in the form of an acid addition salt of hydrochloride.

11. The composition of claim 1, wherein the derivative of Paracetamol is selected from the group consisting of Propacetamol and Phenidine.

12. The composition of claim 1, wherein components (a) and (b) are present in a weight ratio such that the composition will exert a synergistic effect upon administration to a patient.

13. A composition comprising a combination of one or more auxiliary agents and a composition according to claim 1.

14. The composition of claim 13, wherein said composition of claim 13 is in the form of a pharmaceutical dosage formulation comprising a pharmaceutically effective amount of a composition according to claim 1.

15. The composition of claim 14, wherein said pharmaceutical dosage formulation is suitable for oral, intravenous, intraperitoneal, intradermal, intrathekal, intramuscular, intranasal, transmucosal, subcutaneous, or rectal administration.

16. The composition of claim 14, wherein one or both of components (a) and (b) are present in controlled-release form.

17. The composition of claim 14, further comprising caffeine.

18. A method of manufacturing a composition, said method comprising the step of combining one or more auxiliary agents with
(a) at least one 3-(3-Dimethylamino-1-ethyl-2-methyl-propyl)-phenol compound corresponding to formula (I), 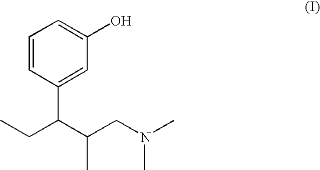
or a solvate or an acid addition salt thereof, and
(b) Paracetamol or a derivative thereof.

19. A method of treating pain in a mammal, said method comprising the step of administering to said mammal a pharmaceutically effective amount of one or more auxiliary agents and a composition comprising:
(a) at least one 3-(3-Dimethylamino-1-ethyl-2-methyl-propyl)-phenol compound corresponding to formula (I), 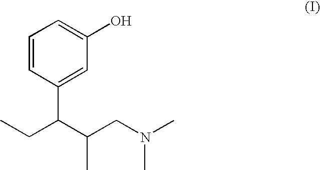
or a solvate or an acid addition salt thereof, and
(b) Paracetamol or a derivative thereof.

20. The method of claim 19, wherein component (a) and (b) of the combination are administered simultaneously.

21. The method of claim 19, wherein component (a) and (b) of the combination are administered sequentially and compound (a) may be administered before or after compound (b).

22. The method of claim 19, wherein compounds (a) or (b) are administered to the mammal by the same administration pathway.

23. The method of claim 19, compounds (a) or (b) are administered to the mammal by a different administration pathway.

24. The method of claim 19, wherein the pain is selected from the group consisting of inflammatory pain, neuropathic pain, acute pain, chronic pain, visceral pain, migraine pain and cancer pain.
